(19) United States
(12) Patent Application Publication
(10) Pub. No.: US 2009/0210950 A1
(43) Pub. Date: Aug. 20, 2009

(54) METHODS FOR SCREENING FOR AGENTS THAT AFFECT DEVELOPMENT

(75) Inventor: Anthony F. Purchio, Taylorsville, UT (US)

Correspondence Address: MORGAN, LEWIS & BOCKIUS LLP (SF), One Market, Spear Street Tower, Suite 2800, San Francisco, CA, 94105, US

(73) Assignee: Numira Biosciences, Inc., Irvine, CA (US)

(21) Appl. No.: 12/389,094

(22) Filed: Feb. 19, 2009

Related U.S. Application Data
(60) Provisional application No. 61/030,149, filed on Feb. 20, 2008.

(51) Int. Cl. G01N 33/53; C12N 15/01; A01K 67/027

(52) U.S. Cl. 800/3; 800/21; 800/18

(57) ABSTRACT

The present invention provides an animal toxicity model that can be used to screen agents that may cause or increase the occurrence of a developmental defect. The invention also provides methods and compositions for creating such an animal toxicity model, as well as methods and kits for using these animal models.

### CROSS REFERENCE TO RELATED APPLICATIONS

This application claims the benefit of priority to U.S. Provisional Patent Application No. 61/030,149, filed on Feb. 20, 2008 which is herein incorporated by reference in its entirety.

### FIELD OF THE INVENTION

The present invention relates generally to screening for agents that have the potential to negatively impact the development of an organism in utero. In particular, the invention provides animal toxicity models that can be used to screen for such agents.

### BACKGROUND OF THE INVENTION

A number of agents found in the environment, in food and beverage products, and in pharmaceuticals have the potential to cause or increase the occurrence of developmental defects. It can be challenging to determine whether a particular agent can have such an effect, however, because developmental studies generally require painstaking experiments at each stage of an organism's development in utero. These studies often require the sacrifice of multiple animals and can also require multiple genetic, physiological and anatomical tests to determine whether an agent has the potential of causing developmental toxicity. Such studies are not amenable to the high throughput screening that is often required when validating a drug or developing safety standards for certain toxins.

### SUMMARY OF THE INVENTION

Accordingly, the present invention provides methods, compositions and systems for screening for agents that have the potential to cause developmental defects.

In one aspect, the invention provides a method of determining whether an agent causes a developmental defect. This method includes the steps of exposing a toxicity mouse model to the agent and detecting a signal in the blood of the toxicity mouse model. In such a method, the presence of the signal identifies the agent as causing developmental toxicity.

In another aspect, the invention provides a method for generating a toxicity mouse model. This method can include the step of crossing a wildtype female with a transgenic male to produce embryos carrying a transgene. In an exemplary aspect, the transgene carried by the embryos includes a BioReporter.

In a further aspect, the invention provides a mouse model for developmental toxicity. The mouse model includes transgenic embryos carried by a wildtype mother. In an exemplary aspect, the transgenic embryos carry a gene encoding a BioReporter.

### DETAILED DESCRIPTION OF THE PREFERRED EMBODIMENTS

### Definitions

The singular forms “a,” “an,” and “the” include plural references, unless the context clearly dictates otherwise. Thus, for example, reference to “an image” encompasses one, two or more images.

As used herein, the term “organism” refers to any living entity comprised of at least one cell. A living organism can be as simple as, for example, a single eukaryotic cell or as complex as a mammal. The term “organism” encompasses naturally occurring as well as synthetic entities produced through a bioengineering method such as genetic engineering.

The term “identifying” (as in “identifying an anatomical feature”) refers to methods of analyzing an object or property, and is meant to include detecting, measuring, analyzing and screening for that object or property.

A “property” is any biological feature that can be detected and measured.

As used herein, the term “tissue” includes cells, tissues, organs, blood and plasma.

A “phenotype” is an observable physical or biochemical characteristic of an organism, as determined by both genetic makeup and environmental influences.

“Manipulation” (as in “manipulation to the animal”) refers to any internal or external procedure applied to a subject. For example, genetic manipulation can include gene therapy, genetic engineering, siRNA/miRNA administration, and transfection. Pharmacological therapy, radiation therapy, and surgery are also included in the term “manipulation”.

The term “expressing” refers to the process of creating and producing a biological feature, including genes, proteins, and physiological characteristics. Expressing a gene includes induction or production of nucleic acids encoding the gene. Expressing a protein includes translation of mRNA to produce protein encoded by a particular gene. “Expressing” also encompasses changes in configuration or structure of molecular, anatomical and cellular structures.

The term “teratogen” refers to an agent that may cause physical defects in the developing embryo or fetus when a pregnant female is exposed to that agent.

The term “agent” refers to any molecule, compound or substance that may cause a detectable effect in the methods, compositions and systems described herein.

The term “developmental toxicity” refers to adverse effects induced during pregnancy, or as a result of parental exposure, that can be manifested at any point in the life span of the organism. The term developmental toxicity encompasses teratogenic/developmental defects.

The term “wildtype” refers the normal phenotype present in a natural or laboratory population as distinct from a mutant type.

### Introduction

The present invention relates to methods, compositions and systems for determining whether an agent affects the development of a fetus. Affecting the development of a fetus can include without limitation: structural defects, physiological and functional defects, and an increased likelihood of developing certain illnesses, including cancer.

In particular, the present invention provides a toxicity animal model that can be used to screen for agents that affect the development of a fetus. In an exemplary embodiment, the toxicity animal model can be used to screen for agents that have some kind of developmental toxicity. In another exemplary embodiment, the toxicity animal model can be used to determine whether an agent causes inflammation in a fetus. In still another exemplary embodiment, the toxicity animal model of the invention can be used to screen for agents that are teratogens or that have teratogenic effects.

In a preferred aspect, the toxicity animal model is a pregnant mouse, where the mother is “wildtype” but the fetuses she carries are transgenic. Such a model is created in an exemplary embodiment by crossing a wildtype female with a transgenic male.

In one aspect, the transgenic fetuses of the model are genetically engineered to express a BioReporter that is linked to a developmental defect. In one embodiment, the BioReporter produces a detectable signal in response to the presence of an agent that causes a developmental defect. Such a detectable signal is in a preferred embodiment released into the bloodstream of the mother and detected from a sample of the mother's blood.

The present invention provides compositions, methods and systems for determining whether an agent has the potential to cause a developmental defect. The invention also provides methods, compositions and systems for generating the toxicity animal models of the invention, and for using these models in high throughput screening methods.

### Toxicity Animal Model

The invention provides a toxicity animal model for the detection and identification of agents that cause developmental defects. Such toxicity animal models may utilize any non-human species amenable to genetic engineering. Preferred models are animals generally used in the art as preclinical animal models for testing of pharmaceuticals and other treatments and for detection of environmental contaminants. Particularly preferred toxicity animal models are mouse models. Although the following description refers to the generation and use of mouse models, it will be appreciated that any transgenic animal model is encompassed by the invention described herein.

In a preferred aspect, the toxicity animal model of the invention is a pregnant mouse, where the mother has a wildtype genome but the fetuses she carries are transgenic. The term “toxicity animal model” refers generally to the pregnant female together with her fetuses. Such an animal model can be generated by crossing a transgenic male with a wildtype female, resulting in fetuses which carry one or more transgenes. The term “fetus” as used herein refers to any prenatal organism between conception and birth which is normally developed in utero. This definition includes a prenatal organism which is first conceived in vitro and later implanted in a uterus. The term “fetus” includes the term “embryo”, and as used herein the terms are used interchangeably unless otherwise indicated.

In one aspect, the transgene carried by the fetuses of the toxicity animal model comprises a BioReporter operationally linked to an inducible promoter. This promoter may be directly or indirectly associated with a developmental defect. As will be appreciated, to be “associated with” a developmental defect can mean that the molecule or agent is a causative factor in that developmental defect. To be “associated with” a developmental defect can also mean that the agent is somehow linked with the developmental defect, but is not necessarily a causative factor. For example, certain genetic markers are associated with certain illnesses, but those markers do not necessarily encode proteins that cause those illnesses. BioReporters of use in the present invention are described in U.S. patent application Ser. No. 11/888,995, filed Aug. 30, 2007, which is herein incorporated by reference in its entirety for all purposes, and in particular for all descriptions, figures and claims related to BioReporters, producing BioReporters and BioReporter systems, detecting BioReporters, and using BioReporters in assays.

In one aspect, the toxicity animal models of the invention comprise a BioReporter operably linked to a promoter that can be induced by a teratogen. In another aspect, the promoter can be induced by another molecule which is itself activated by a teratogen.

As will be appreciated, any promoter can be used to drive transcription of a BioReporter in toxicity animal models of the invention. In an exemplary embodiment, the promoter is associated with one or more members of the p450 enzyme family in combination with a BioReporter. The p450 enzymes are responsible for almost all of the detoxification functions that take place in the body.

In one embodiment, promoters and/or BioReporters used in the animal toxicity models of the invention are induced by transcription factors. In a preferred embodiment, these transcription factors are associated with one or more developmental defects.

In one exemplary embodiment, an agent with teratogenic effects will bind to a transcription factor, causing that transcription factor to translocate to the nucleus and induce the promoter, which in turn drives the transcription of the BioReporter. Such a transcription factor may be known to mediate pleiotropic biological responses, including biological responses that cause structural and functional birth defects.

Transcription factors that can be used in the animal toxicity models of the invention can include without limitation: MSX transcription factors (including MSX-1 and MSX-2), TWIST, GATA-1, GATA-4, Nuclear Factor of activated T cells (NF-AT), Err2, Gcm1, winged helix/forkhead transcription factor, serum response factor (SRF), neuronal stem cell leukemia (NSCL) basic helix-loop-helix factors, T-box transcription factor, HAND2, Gfi1, mitochondrial transcription factor A (TFAM), regulatory factor X (RFX), TEAD2, Mesp1, Coup-TFII, and WT1. In addition, toxicity animal models of the invention may utilize cofactors and/or co-regulators of transcription factors, including GATA-4.

### BioReporters

In one aspect, the invention provides toxicity animal models that comprise BioReporters. BioReporters are exogenous molecules expressed in or produced by an organism. The organism expresses or produces the BioReporter as a result of genetic engineering, gene therapy, incorporation of a genetically altered cell or cellular product into an organism, xenograft of cells, tissues and organs from one organism to another, as well as by other methods known in the art for causing an organism to express and/or produce an exogenous molecule or biological characteristic. In an exemplary embodiment, a toxicity animal model of the invention comprises a BioReporter that produces a detectable signal in response to an agent that has the potential to cause a developmental defect.

BioReporters and/or BioReporter signals encompass any molecule or biological feature that can be manipulated, induced, detected, and/or quantified. In a preferred aspect, BioReporters are proteins, carbohydrates, nucleic acids, lipids, metabolites, carbohydrates, salts, and small molecules. In one example, such BioReporters are released from the transgenic embryos into the mother's blood stream. The presence of such BioReporter signals can then be detected using a simple blood test.

BioReporters and/or BioReporter signals may also include other biological features, such as anatomical characteristics (for example, organ structure, shape and condition), cellular components (such as mitochondria and chloroplasts), and physiological features (such as blood pressure, heart rate, and respiratory rate).

BioReporters of the invention encompass known biomarkers as well as newly generated and spontaneously occurring biomarkers which have been identified and analyzed using methods of the present invention. In a preferred embodiment, BioReporters of the invention produce a detectable signal, such as a secreted molecule or an optical signal. One example of a detectable BioReporter signal is secreted alkaline phosphatase (SEAP-Clontech). The secreted expression of SEAP is indicative of tumor burden and can be used as an evaluation tool for anticancer drug efficacy. In another exemplary embodiment, the BioReporter is firefly luciferase, which creates a luminescent signal upon application of luciferin to the organism expressing the luciferase gene.

In a preferred aspect of the invention, BioReporters possess properties which are detectable and/or quantifiable. The properties exhibited by a BioReporter will depend on the type of BioReporter. For example, BioReporters which are molecules such as proteins and nucleic acids will have properties that include expression level, patterns of expression, localization to particular tissues, and ability to bind to or be bound by substrates. BioReporters may also have properties which include anatomical characteristics, cellular shape and structure, intracellular structures, and physiological features such as blood pressure, skin color, respiratory rate, heart rate, and blood oxygen level.

In a particularly preferred embodiment, BioReporters are associated with a detectable signal. In one embodiment, the BioReporter itself produces a signal. For example, the BioReporter can be a fluorescent or luminescent protein, such as a fluorescent protein (e.g., green fluorescent protein (GFP) or blue fluorescent protein (BFP)) or luciferase. In another embodiment, the BioReporter induces a signal, for example by binding to a receptor which in turn activates the production of a secreted protein. The detectable signal produced by or induced by the BioReporter can be optical signals and secreted signals. Such detectable signals are also referred to herein as “BioReporter signals”.

In one embodiment, BioReporters are labeled with another molecule that produces a detectable signal. Such labeling may be achieved by covalently or non-covalently joining a moiety which directly or indirectly provides a detectable signal. BioReporters can be labeled either directly or indirectly. Possibilities for direct labeling include label groups: radiolabels such as ¹²⁵I, enzymes (U.S. Pat. No. 3,645,090) such as peroxidase and alkaline phosphatase, and fluorescent labels (U.S. Pat. No. 3,940,475) capable of monitoring the change in fluorescence intensity, wavelength shift, or fluorescence polarization. Possibilities for indirect labeling include biotinylation of one constituent followed by binding to avidin coupled to one of the above label groups. A label may be detectable by spectroscopic, photochemical, biochemical, immunochemical, electrical, optical or chemical means. Examples include, but are not limited to, magnetic beads (e.g., Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase, and others commonly used in an ELISA), and colorimetric labels such as colloidal gold or colored glass or plastic (e.g., polystyrene, polypropylene, latex, etc.) beads.

In any specific embodiment of the invention, an exemplary BioReporter can be without limitation: secreted alkaline phosphatase, firefly luciferase, Gaussia luciferase, red fluorescent protein, green fluorescent protein, beta-2-microglobulin, allantoin (including allantoin produced by exogenous xanthine oxidase), sialylated neural cell adhesion molecule (NCAM), Pax7 (paired box gene 7), and beta-human chorionic goanotropin (B-HCG).

In one embodiment, toxicity animal models of the invention utilize an aryl hydrocarbon receptor (AhR or dioxin receptor) in combination with a BioReporter. It has been shown that mice lacking this receptor fail to show a teratogenic response to certain environmental contaminants (Yamashita et al., Genes Cells. (1997) October; 2(10):645-54). In one possible mechanism of action, an agent that causes developmental defects binds to the AhR, resulting in translocation of the drug-receptor complex to the nucleus. Upon entering the nucleus, the drug-receptor complex binds to a xenobiotic response element that is operationally linked to a gene encoding a BioReporter. In a preferred embodiment, the xenobiotic response element is contained within or is adjacent to a promoter, such that binding of the drug-receptor complex to the xenobiotic response element causes transcription of the BioReporter gene, resulting in expression of the BioReporter, which can then be detected using methods as described herein.

### Making the Animal Model

Transgenic mice are achieved routinely in the art using the technique of microinjection, as described in U.S. Pat. No. 4,736,866 and by B. Hogan et al. in “Manipulating the Mouse Embryo: A Laboratory Manual”, Ed. 2, pp. 89 204. Plainview, N.Y.: Cold Spring Harbor Laboratory, USA (1995). Further methods for the production of a transgenic non-human animal, for example a transgenic mouse, comprise introduction of a targeting vector into a germ cell, an embryonic cell, stem cell or an egg or a cell derived therefrom.

In an exemplary embodiment, toxicity animal models of the invention are created by crossing a wildtype female with a transgenic male, resulting in a pregnant wildtype female carrying transgenic embryos.

In one embodiment, the animal toxicity models of the invention comprise BioReporter systems that include Cre-inducible reporter mouse lines, which can be used for identifying cells of a specific lineage as well as all of the cells that are derived from the cells that were originally (genetically) marked. The DNA recombinase Cre can permanently rearrange genomic DNA where short 34 base pair LoxP sites have been transgenically engineered into mouse loci. In conditional mouse lines, Cre can mediate the inactivation of genes (i.e., conditional knockout alleles) or the activation of genes (i.e., conditional knock-in alleles & conditional reporter alleles). Such mouse lines can be established using methods known in the art. (see eg., Brocard et al., (1997) PNAS, 94:14559-14563; Lyons et al., (2003), Cancer Res, 63:7042-7046, Vasioukhin et al., (1999), PNAS, 96:8551-8556, which are hereby incorporated by reference in their entirety).

### Using the Animal Model

Toxicity animal models of the invention are particularly useful in studies requiring multiple datapoints along various time points during gestation. For example, in one aspect the invention provides a toxicity animal model comprising a BioReporter that secretes a protein into the mother's bloodstream in response to an agent. In such a model, a simple blood test can be used to detect the presence of the protein and thus identify that agent as having a teratogenic effect. Using a blood test provides a method of detecting the BioReporter signal without requiring sacrifice of the animal, which allows multiple tests to be conducted throughout the development of the fetuses. The ability to monitor effects of an agent over a period of time can provide data as to the levels of an agent required for a teratogenic effect to occur, and can also provide data as to the developmental stage at which a particular agent exerts its teratogenic effect. Another advantage of the toxicity animal models of the invention is that if the fetuses can be carried to term, additional studies can be made of the pups, thus providing further data on any teratogenic effects of the applied agent(s).

In addition to blood tests, other methods and/or samples from the mother can be used to detect a BioReporter signal generated in response to an agent that may cause a developmental defect. The samples used in these methods can include plasma, biological fluids and cells, and mixtures thereof. Physiological measurements, such as blood pressure, skin color, respiratory rate, heart rate, and blood oxygen level can also be used to detect a BioReporter signal.

As will be appreciated by those in the art, the present invention is not limited in the type, characteristic, or form of the methods used to detect and analyze properties of BioReporters and biomarkers, and the method chosen to detect and analyze a particular property will depend on the type of BioReporter/biomarker and the property being studied. For example, if the biomarker is a nucleic acid and the property being analyzed is expression level, then methods of detection and analysis can utilize (without limitation) microarrays, polymerase chain reaction (PCR), electrophoresis, Northern or Southern blots, and spectroscopy. Such techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference).

Similarly, other kinds of biomarkers and BioReporters can be detected and analyzed using appropriate methods known in the art. For example, calorimetric assays using dyes are widely available. Alternatively, detection may be accomplished spectroscopically. Spectroscopic detectors rely on a change in refractive index; ultraviolet and/or visible light absorption, or fluorescence after excitation with a suitable wavelength to detect reaction components. Exemplary detection methods include fluorimetry, absorbance, reflectance, and transmittance spectroscopy. Changes in birefringence, refractive index, or diffraction may also be used to monitor complex formation or reaction progression. Particularly useful techniques for detecting molecular interactions include surface plasmon resonance, ellipsometry, resonant mirror techniques, grating-coupled waveguide techniques, and multi-polar resonance spectroscopy. These techniques and others are well known and can readily be applied to the present invention by one skilled in the art, without undue experimentation.

In a preferred aspect, imaging is used to detect the BioReporter signal generated in response to a teratogenic agent. For example, virtual histology studies can be used to image the embryos in utero. Virtual histology methods are described in copending application number PCT/US07/02264, which is hereby incorporated in its entirety for all purposes, and in particular for teaching of methods of whole organism imaging.

In vivo monitoring can also be carried out using, for example, bioluminescence imaging, planar gamma camera imaging, SPECT imaging, light-based imaging, magnetic resonance imaging and spectroscopy, fluorescence imaging (especially in the near infrared), diffuse optical tomography, ultrasonography (including untargeted microbubble contrast, and targeted microbubble contrast), PET imaging, fluorescence correlation spectroscopy, in vivo two-photon microscopy, optical coherence tomography, speckle microscopy, small molecule reporters, nanocrystal labeling and second harmonic imaging, as well as others. Massoud et al. provide a detailed review of molecular imaging technologies (Genes and Development, 17:545-580, 2003), which is herein incorporated in its entirety for its teaching regarding molecular imaging.

Agents that are being screened for potential teratogenic effects can be provided to the toxicity animal model by any method that allows the agent to eventually enter the uterus and interact with the transgenic embryos. In one exemplary embodiment, the agent is introduced into the mother's bloodstream, and thus is provided to the developing embryos through the umbilical cord. The agent may be provided to the mother through ingestion of food or water. The agent may also be injected into or inhaled by the mother. The agent may also be absorbed through the mother's skin and/or fur. In some embodiments, the agent may be directly applied to the uterus and/or the embryos carried in the uterus, for example, through an intrauterine injection.

### Applications

In a preferred aspect, the toxicity animal models of the invention are used to screen agents to determine if they have the potential to cause developmental defects. As will be appreciated, any agent can be screened for potential effects on development using the invention, including without limitation pharmaceuticals, nutraceuticals, biologics, chemicals, and the like. In an exemplary aspect, the toxicity mouse model is exposed to the agent, and a signal is detected in the blood of the mouse model. The presence of such a signal identifies the agent as causing developmental toxicity. In a preferred embodiment, the toxicity mouse model is a pregnant wildtype mother carrying transgenic embryos, and detecting the presence of a signal includes conducting a blood test on the mother using techniques known in the art.

In one aspect, the toxicity animal model of the invention is used to monitor inflammation in a fetus. In such an exemplary aspect, a BioReporter, such as the gene for human chorionic gonadotropin (hCG) is operably linked to a promoter that drives inflammatory genes, such as interleukins, inductible nitric oxide synthase (iNOS), and tumor necrosis factor (TNF). An agent that has the potential to cause inflammation in a fetus would activate such promoters, which would in turn drive the transcription of the detectable BioReporter. Detection of the signal from the BioReporter would provide an identification of the inflammatory potential of the agent, and would also allow for long-term monitoring of any inflammation in the fetus throughout its development.

Similarly, in other aspects, the toxicity animal model of the invention can be used to detect and monitor any developmental toxicity caused by an agent. As will be appreciated, the animal model can be designed with the combination of BioReporters and/or promoters appropriate to detect and monitor the effects of a particular type of developmental toxicity.

The invention also provides systems, including kits which can be used to screen for agents that have the potential to cause developmental defects. In one aspect, the invention provides a kit that includes an animal toxicity model as described herein, tools and apparatuses for obtaining a sample from the animal toxicity model, and tools, compositions and instructions for detecting the presence of a BioReporter and/or a BioReporter signal in the sample.

In one aspect, the animal toxicity model of the invention is used as a high throughput screen for agents that can cause a biological, physiological and/or anatomical effect on a fetus, wherein that effect is detected by the presence of a BioReporter, as described herein. In such a high throughput screen, multiple animals exposed to the same or different agents can be screened at the same time, for example, using the virtual histology imaging methods described herein. In another aspect, the invention utilizes high throughput analyses such as molecular or protein arrays to test for the presence of multiple BioReporter signals from animal models exposed to one or more agents.

Images acquired in assays and screens described herein and known in the art can be analyzed using methods known in the art as well as methods described in U.S. patent application Ser. No. 11/839,414, filed Aug. 15, 2007, which is herein incorporated by reference in its entirety for all purposes and in particular for all teachings, descriptions, figures and claims related to the analysis of images and the development and use of libraries formed from compiling such images and data from analysis of such images.

As will be appreciated, the invention is not limited to only the exemplary aspects and embodiments described herein.

All patents, patent applications, and other publications cited in this application are incorporated by reference in their entirety.

We claim:

1. A method of determining whether an agent causes a developmental defect, the method comprising:
(a) exposing a toxicity mouse model to the agent;
(b) detecting a signal in the blood of the toxicity mouse model,
wherein the presence of the signal identifies the agent as causing developmental toxicity.

2. The method of claim 1, wherein the toxicity mouse model comprises a pregnant wildtype mother carrying transgenic embryos, and the detecting step (b) comprises conducting a blood test on the wildtype mother.

3. The method of claim 2, wherein the transgenic embryos comprise a BioReporter.

4. The method of claim 3, wherein the BioReporter comprises an inducible promoter operably linked to a gene that produces the signal.

5. The method of claim 4, wherein the inducible promoter is associated with a developmental defect.

6. The method of claim 5, wherein the inducible promoter comprises a binding site for an aryl hydrocarbon receptor.

7. The method of claim 4, wherein the gene encodes for a soluble protein.

8. The method of claim 7, wherein the soluble protein is released into the bloodstream of the wildtype mother.

9. The method of claim 7, wherein the soluble protein is hCG.

10. The method of claim 9, wherein an upregulation of hCG expression identifies the agent as causing developmental toxicity.

11. The method of claim 10, wherein the upregulation is detected in a blood test of the wildtype mother.

12. A method for generating a toxicity mouse model, the method comprising crossing a wildtype female with a transgenic male to produce embryos carrying a transgene, wherein the transgene comprises a BioReporter.

13. The method of claim 12, wherein the BioReporter comprises an inducible promoter operably linked to a gene encoding for hCG.

14. A mouse model for developmental toxicity, wherein the mouse model comprises transgenic embryos carried by a wildtype mother, wherein the transgenic embryos comprise a BioReporter.

15. The model of claim 14, wherein the BioReporter comprises a promoter operably linked to a gene encoding for a soluble protein.

16. The model of claim 15, wherein the soluble protein is released into the bloodstream of the wildtype mother.

17. The model of claim 16, wherein the soluble protein is detected using a blood test from the wildtype mother.

18. The model of claim 15, wherein the soluble protein is hCG.
